(19) United States
(12) Patent Application Publication
(10) Pub. No.: US 2007/0021806 A1
(43) Pub. Date: Jan. 25, 2007

(54) Controllable light therapy apparatus, assembly including the same, and method of operating associated thereto

(76) Inventors: Charles Mercier, Mont-Saint-Hilaire (CA); Jennifer Brodeur, Mont-Saint-Hilaire (CA); Tony Mori, Sainte-Therese (CA)

Correspondence Address: ROBIC, CENTRE CDP CAPITAL, 1001, VICTORIA SQUARE - BLOC E - 8TH FLOOR, MONTREAL, QC, H2Z 2B7, CA

(21) Appl. No.: 10/558,415

(22) PCT Filed: May 28, 2004
(86) PCT No.: PCT/CA04/00802

§ 371 Date: Nov. 28, 2005

(30) Foreign Application Priority Data
May 28, 2003 (CA) 2,429,906

(51) Int. Cl. A61N 5/06

(52) U.S. Cl. 607/88

(57) ABSTRACT

An apparatus for treating the skin of a patient by means of light therapy. The apparatus includes a base, a light emitting surface, moving means, and control means. The light emitting surface includes an array of light emitting sources, each source being configured for emitting different rays of light at different wavelengths. The moving means are used for controllably and remotely moving the light emitting surface with respect of the base along a predetermined path and at a predetermined rate. The control means are used for controlling the light emitting sources so that each source emits a given ray of light at a given wavelength, and for controlling the moving means so that the light emitting surface is moved to treat the skin of a patient. Also described is an assembly including the apparatus, as well as a method of operating the same.

### FIELD OF THE INVENTION

The present invention relates to a light therapy apparatus. More particularly, the present invention relates to an apparatus with a controllably and remotely movable light emitting surface used for treating the skin of a patient by means of light therapy. The present invention also relates to an assembly including the apparatus, and to a method of operating the same.

### BACKGROUND OF THE INVENTION

Known in the art are various light therapy devices used for the treatment of various skin conditions, including: eczema, psoriasis, acne, contact dermatitis, and the like. Research has proven that living cells in skin respond to light. In fact, it has been shown that the response of a cell differs depending on the wavelength emitted from the light source to which it is exposed.

Light sources used for such therapy are generally of two types: chromatically filtered incadescent light sources, and Light Emitting Diodes, also known as LEDs. In both cases, the light source emits light with a particular wavelength range. The range will vary depending on the quality of the chemicals used and the assembly process of the light source. Usually, the wavelength range follows a normal distribution with a span of within 10 nanometers of the dominant wavelength. Most of the time, this variation cannot be perceived by the human eye. In the context of the following description, this variation within the light source will not be taken into account, and therefore, light will be referred to as being of a “single wavelength”, for sake of simplicity and convenience.

Also known in the art are various types of light therapy devices used for medical and esthetic applications. These apparatuses all use a light source which is directed toward the patient in order to treat different skin conditions. These apparatuses use different configurations to achieve the desired light exposure.

The first and most simple known configuration is that of a fixed incandescent light source. Usually, this type of apparatus consists of a panel which incorporates the light source. This panel is positioned using either a floor or a table stand. These types of units emit light of a specific wavelength constantly for the duration of the treatment. This type of apparatus is disadvantageous because the fact of being exposed in front of a fixed panel throughout the entire duration of the treatment is fairly uncomfortable for a patient, and furthermore, the panel must be manually displaced by an operator when treating other portions of the skin of the patient, and/or other body parts.

The second known configuration is that of a panel incorporating a plurality of LEDs of a same composition. This panel is also positioned using either a floor or a table stand. This type of unit emits light of a specific wavelength which can be constant or pulsed for the duration of the treatment. This second type of apparatus is also disadvantageous for reasons similar to the ones mentioned above. Furthermore, it is also worth mentioning that there are drawbacks associated with fixed light sources being emitted constantly which result from the fact that, when skin is exposed to a constant intensity, the skin becomes saturated after a certain time, and thus does not respond anymore to the light therapy treatment. There are also drawbacks associated with fixed light sources being emitted in a pulsating manner which result from the fact that their lifespan is fairly shorten as a result of the alternation of the operation of the components. Indeed, the on-and-off action of the light emitting sources is strenuous on the components and thus causes them to fail or need to be replaced more frequently, which is undesirable for obvious reasons known in the art.

The third known configuration resembles the above-mentioned second configuration except that it incorporates LEDs of different compositions, usually two, to enable the device to emit different wavelengths. The wavelength being emitted is controlled by the user or by the device itself through the use of an electronic controller. The light emitted can be constant or pulsed. Thus, similar drawbacks to the above-described configuration apply here also.

The fourth known configuration is that of a light probe that is moved manually by the technician or the patient to the desired position in order to perform the treatment. These probes typically use one or a plurality of LEDs of the same composition as a light source. This type of unit emits light of a specific wavelength which can be constant or pulsed for the duration of the treatment. However, this type of device needs to be continuously manipulated by an operator treating the skin, which therefore requires the constant presence and/or manoeuvring of the operator throughout the entire duration of the treatment, which is also disadvantageous.

The fifth known configuration resembles the above-mentioned fourth configuration except that it incorporates LEDs of different compositions, usually two, to enable the device to emit different wavelengths. The wavelength being emitted can be constant or pulsed. They are controlled by the user or by the device itself through the use of an electronic controller. Thus, similar drawbacks to the above-described configurations apply here also.

The sixth known configuration is a light probe that uses a white light source that is filtered to achieve the desired color. This is accomplished through the use of chromatic filters which are put in place by the technician. This type of probe can be moved manually or held in place by a stand. Thus, similar drawbacks to the above-described configurations apply here also.

Thus, as mentioned above, the problems associated with the above-described light therapy apparatuses are several, namely: a) they are very limited in the different colors (i.e. wavelengths) that they can emit; and b) they are all to be displaced manually and/or fixed in place using a stand. Thus, the treatments that can be performed with these apparatuses are very basic, namely due to the restrictions in available wavelengths used to perform the treatment and/or to the non-versatility of the light emitting surface, which requires to be manually operated in order to be displaced or is limitedly kept fixed in place when held on a stand.

Hence, in light of the above-discussed, there is a need for an improved light therapy apparatus which, by virtue of its design and components, would be able to overcome some of the aforementioned prior art problems.

### SUMMARY OF THE INVENTION

An object of the present invention is to provide a light therapy apparatus which satisfies some of the above-mentioned needs, and which is thus an improvement over related light therapy apparatuses known in the prior art.

The present invention, as will be explained hereinbelow, is particularly advantageous because it is devised to meet the various needs of the skin care industry.

In accordance with the present invention, there is provided an apparatus for treating the skin of a patient, the apparatus comprising:

a base;

a light emitting surface including an array of light emitting sources, each source being configured for emitting different rays of light at different wavelengths;

moving means for controllably and remotely moving the light emitting surface with respect to the base along a predetermined path and at a predetermined rate; and

control means for controlling the light emitting sources so that each source emits a given ray of light at a given wavelength, and for controlling the moving means so that the light emitting surface is moved to treat the skin of the patient.

Preferably, as will be explained in greater detail hereinbelow, the moving means comprise a movable arm having a first portion along which extends the light emitting surface, and a second portion having an end pivotally connected to the base. Preferably also, the moving means comprise a servomotor and a gearbox, the gearbox being operatively connected to the end of the arm pivotally connected to the base, and the servomotor being operatively connected to the gearbox for driving the same and thereby moving the arm. Preferably, the gearbox is a planetary gearbox, the servomotor is connected to the control means for receiving control signals from the same, and the arm, also referred to as a light emitting arm, is substantially L-shaped.

Preferably also, the apparatus comprises an optical lens placed over the light emitting surface, the optical lens being shaped and sized for redirecting the rays of light emitted by the light emitting sources. The optical lens is preferably shaped and sized for redirecting the rays of light emitted by the light emitting sources in a direction substantially perpendicular with respect to the light emitting surface, and for concentrating the rays of light onto the skin to be treated.

Preferably also, the array of light emitting sources comprises a least one row and a plurality of transversal columns, and comprises a plurality of sections, each section being controllable independently by the control means in order to vary a light output from each section.

Alternatively, the moving means may simply comprise a fixed panel where lies the array of light emitting sources, and the moving means may consist of a sequential modulation from the control means of the light emitting sources along the panel for moving the light emitting surface with respect to said panel. Other suitable movable means are intended according to the present invention.

The light emitting sources according to the present invention are preferably light emitting diodes.

Preferably also, the control means comprises a control panel hingedly connected to a top portion of the base, and being tiltable between opposite left and right sides of the base.

According to another aspect of the present invention, there is also provided an assembly including an apparatus such as the one described herein for treating the skin of a patient by means of light therapy.

According to yet another aspect of the present invention, there is also provided a method of operating an apparatus such as the one described herein.

The objects, advantages and other features of the present invention will become more apparent upon reading of the following non-restrictive description of a preferred embodiment thereof, given for the purpose of exemplification, only with reference to the accompanying drawings.

### BRIEF DESCRIPTION OF THE DRAWINGS

FIG. 1 is a perspective view of a controllable light therapy apparatus according to the preferred embodiment of the present invention, the preferred position of a patient being shown in relation to the apparatus.

FIG. 2 is a partial side view of what is shown in FIG. 1.

FIG. 3 is a top plan view of the controllable light therapy apparatus shown in FIG. 1.

FIG. 4 is another top plan view of the controllable light therapy apparatus shown in FIG. 3, the apparatus being shown now with its control panel and motor cover removed to expose the motor, gearbox and power supply of the apparatus.

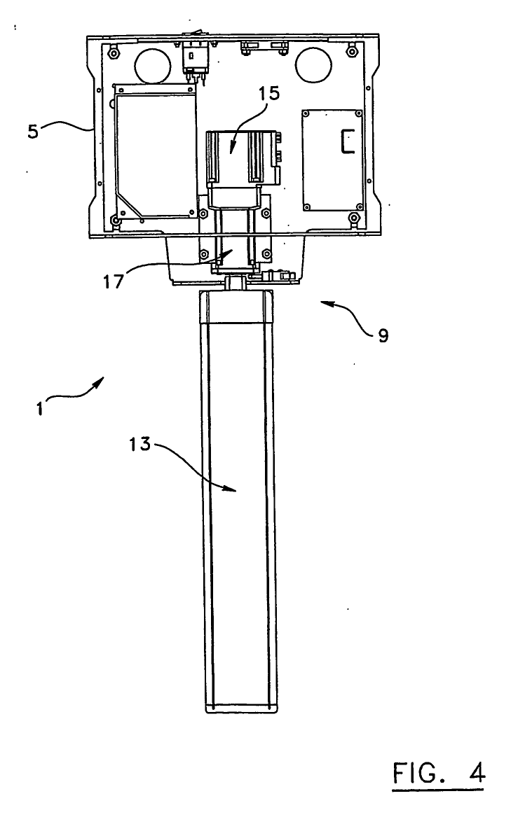

FIG. 5 is a front plan view of the controllable light therapy apparatus shown in FIG. 1, the apparatus being shown with its light emitting arm positioned at an angle of about sixty degrees with respect to a vertical plane.

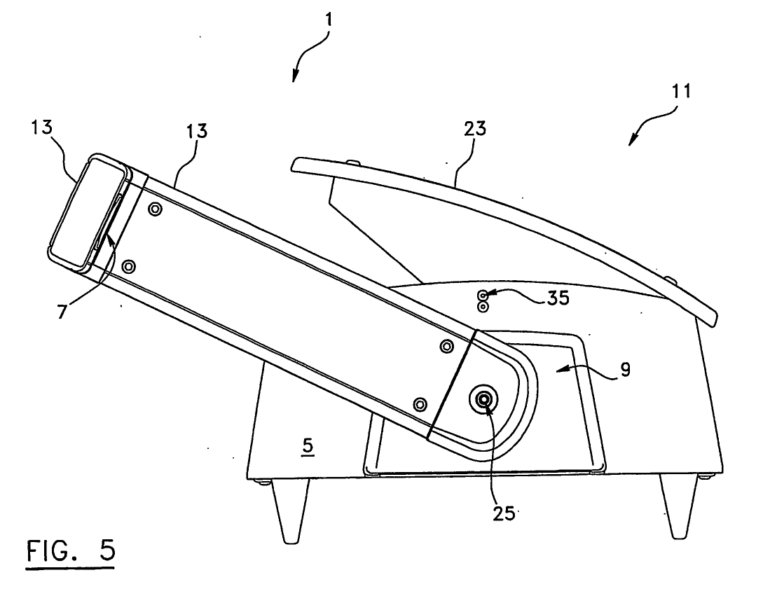

FIG. 6 is another front view of the controllable light therapy apparatus shown in FIG. 5, the apparatus being shown now with its light emitting arm positioned at an angle of about zero degrees.

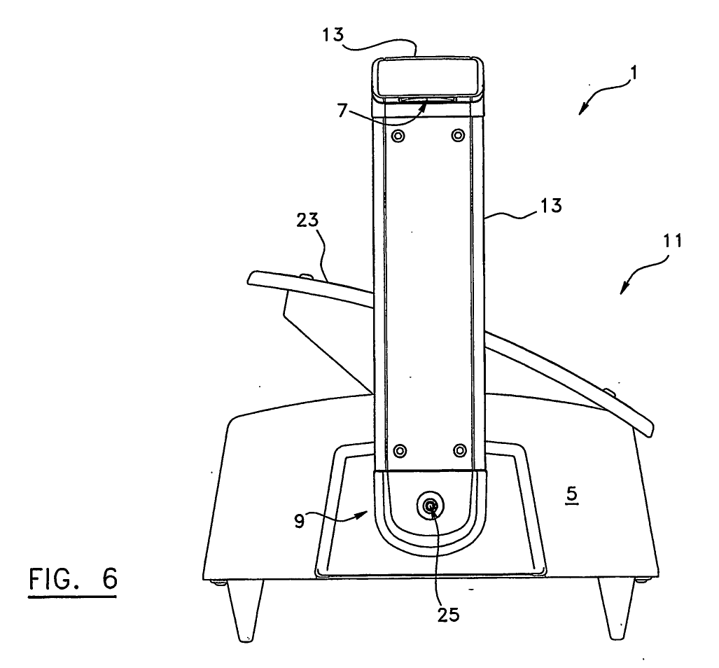

FIG. 7 is another front view of the controllable light therapy apparatus shown in FIG. 6, the apparatus being shown now with its light emitting arm positioned at an angle of about minus sixty degrees.

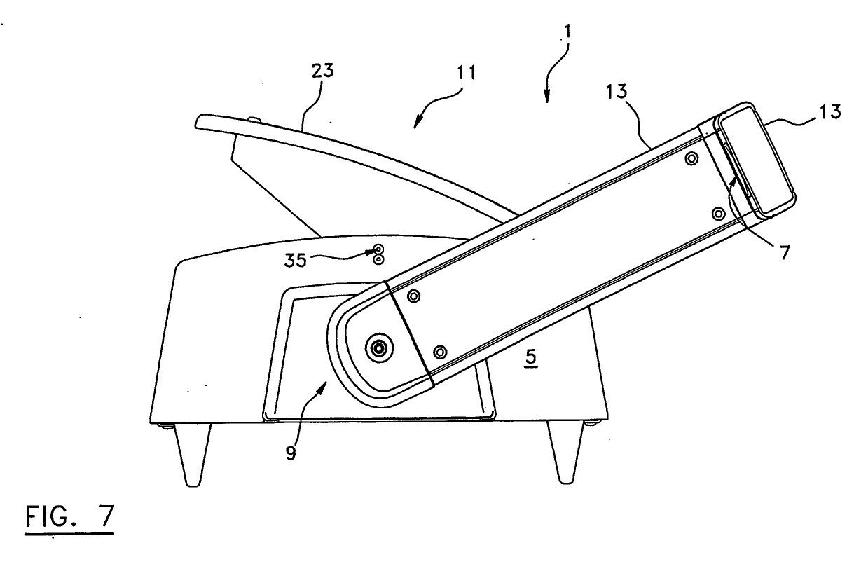

FIG. 8 is a bottom plan view of the controllable light therapy apparatus shown in FIG. 1, said view showing the array of LEDs located in the light emitting surface of the arm.

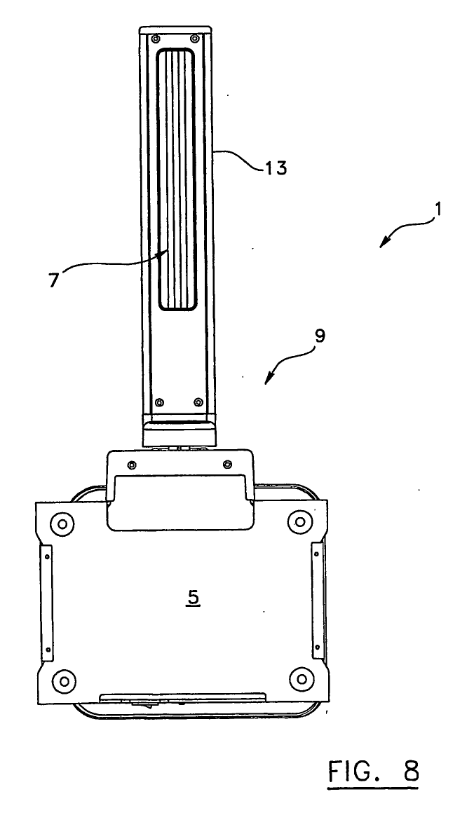

FIG. 9 is a sectional view of the light emitting arm shown in FIG. 8, this view showing the LEDs and the optical lens used to concentrate the rays of light.

FIG. 10 is a rear view of the controllable light therapy apparatus shown in FIG. 1, the apparatus being shown with the control panel positioned to be used on the right side of the apparatus.

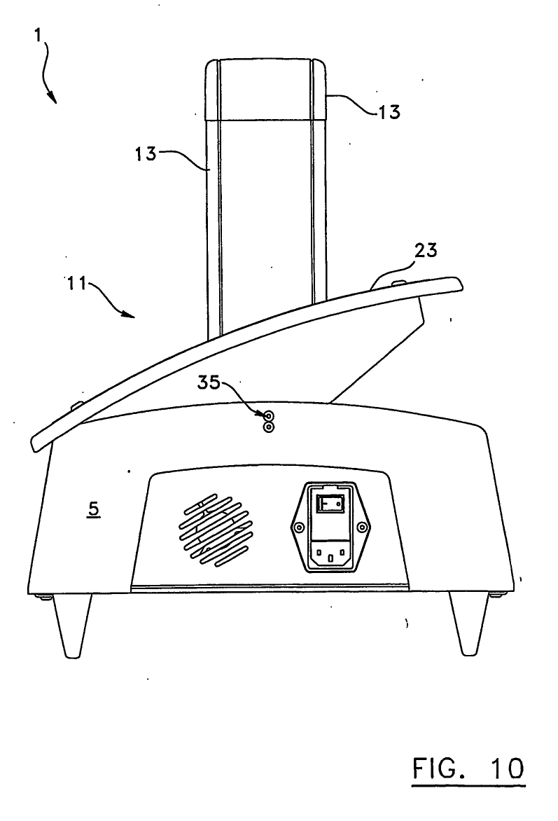

FIG. 11 is another rear view of the controllable light therapy apparatus shown in FIG. 10, the apparatus being shown now with its control panel positioned to be used on the left side of the apparatus.

FIG. 12 is a perspective view of the controllable light therapy apparatus of FIG. 1 being shown resting on a base.

### DETAILED DESCRIPTION OF A PREFERRED EMBODIMENT OF THE INVENTION

In the following description, the same numerical references refer to similar elements. The embodiments shown in the figures are preferred.

Moreover, although the present invention was primarily designed for skin treatment of a patient, it may be used with other types of objects and in other fields, as apparent to a person skilled in the art. For this reason, expressions such as “skin”, “treatment”, “patient”, etc. used herein should not be taken as to limit the scope of the present invention and include all other kinds of individuals, animals, or items with which the present invention could be used and may be useful.

Moreover, in the context of the present invention, the expressions “light”, “source”, “wavelength”, “beam” and any other equivalent expression and/or compound words thereof known in the art will be used interchangeably. Furthermore, the same applies for any other mutually equivalent expressions, such as “device” and “apparatus”, as well as “patient”, “individual” and “person” for example, as also apparent to a person skilled in the art.

In addition, although the preferred embodiment of the present invention as illustrated in the accompanying drawings comprises various components such as an arm, a gearbox, an optical lens, a control panel, etc., and although the preferred embodiment of the light therapy apparatus 1 as shown consists of certain geometrical configurations as explained and illustrated herein, not all of these components and geometries are essential to the invention and thus should not be taken in their restrictive sense, i.e. should not be taken as to limit the scope of the present invention. It is to be understood, as also apparent to a person skilled in the art, that other suitable components and cooperations thereinbetween, as well as other suitable geometrical configurations may be used for the light therapy apparatus 1 and corresponding parts according to the present invention, as briefly explained and inferred herein, without departing from the scope of the invention.

Broadly described, the light therapy apparatus 1 according to the present invention, as illustrated in the accompanying drawings, relates to an apparatus 1 for treating the skin of a patient 3 by means of light therapy. The apparatus 1 includes a base 5, a light emitting surface 7, moving means 9, and control means 11. The light emitting surface 7 includes an array of light emitting sources, each source being configured for emitting different rays of light at different wavelengths. The moving means 9 are used for controllably and remotely moving the light emitting surface 7 with respect of the base 5 along a predetermined path and at a predetermined rate. The control means 11 are used for controlling the light emitting sources so that each source emits a given ray of light at a given wavelength, and for controlling the moving means 9 so that the light emitting surface 7 is moved to treat the skin of the patient 3. According to the present invention, there is also provided an assembly including the apparatus 1, as well as a method of operating the same.

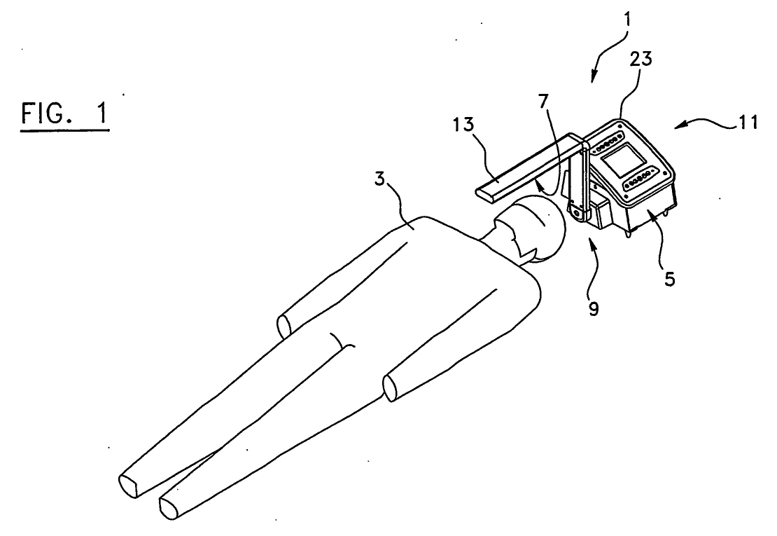

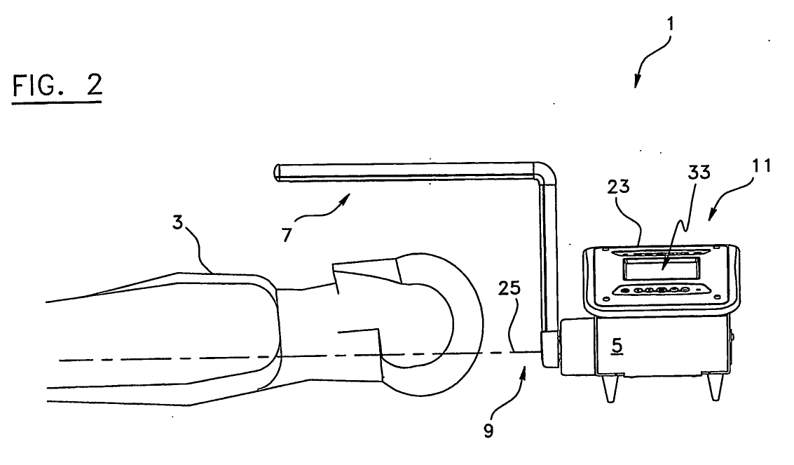

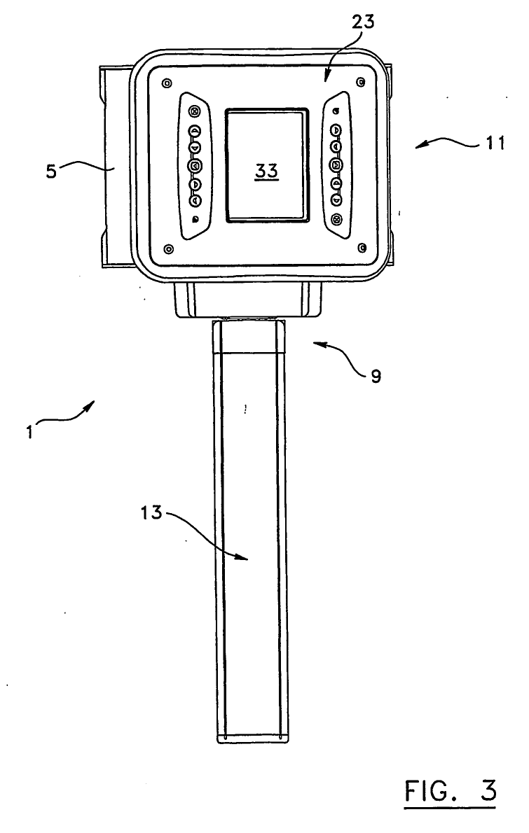

Preferably, as better shown in FIGS. 1 and 2, the moving means 9 comprise a movable arm 13 having a first portion along which extends the light emitting surface, and a second portion having an end pivotally connected to the base. Preferably also, as can be easily understood from FIG. 3, the moving means 9 comprise a servomotor 15 and a gearbox 17, the gearbox 17 being operatively connected to the end of the arm 13 pivotally connected to the base 5, and the servomotor 15 being operatively connected to the gearbox 17 for driving the same and thereby moving the arm. Preferably, the gearbox 17 is a planetary gearbox 17, the servomotor 15 is connected to the control means 11 for receiving control signals from the same, and the arm 13, also referred to as “light emitting arm” 13, is substantially L-shaped, as better shown in FIG. 2. It is worth mentioning that according to the present invention, the arm 13 may take on other suitable shapes and dimensions, depending on the intended use of the apparatus 1. Indeed, instead of rotating with respect to the base 5, the arm 13 could for example be designed to translate with respect to the base (and thus the patient 3), and/or displace itself along other suitable guiding means, as apparent to a person skilled in the art. The control means 11 preferably comprise at least one microprocessor, as will be explained hereinbelow.

Alternatively, as suggested earlier, the moving means 9 may also take on other suitable embodiments, as will be referred hereinbelow, and as apparent to a person skilled in the art. For example, the moving means 9, instead of being “moving means” per se, may simply comprise a fixed panel (not shown) where lies the array of light emitting sources, and the moving means 9 may thus consist of a sequential modulation from the control means 11 of the light emitting sources along the panel for moving the light emitting surface 7 with respect to said panel. Other suitable movable means 9 are intended according to the present invention, as also apparent to a person skilled in the art.

Preferably also, the apparatus 1 comprises an optical lens 19 placed over the light emitting surface 7, the optical lens being shaped and sized for redirecting the rays of light emitted by the light emitting sources. The optical lens 19 is preferably shaped and sized for redirecting the rays of light emitted by the light emitting sources in a direction substantially perpendicular with respect to the light emitting surface 7, and preferably, for concentrating the rays of light onto the skin to be treated, so as to not require the light emitting surface 7 to be too close from the skin to be treated, without compromising treatment effectiveness, thereby allowing an advantageous buffer zone between the skin of the patient 3 and the light emitting arm 13.

Preferably also, the array of light emitting sources comprises a least one row and a plurality of transversal columns, and comprises a plurality of sections, each section being controllable independently by the control means 11 in order to vary a light output from each section. The apparatus preferably comprises three of such sections.

The light emitting sources according to the present invention are preferably light emitting diodes 21.

Preferably also, the control means 11 comprises a control panel 23 hingedly connected to a top portion of the base 5, and being tiltable between opposite left and right sides of the base, as can be easily understood from the accompanying drawings and as will be explained in greater detail below.

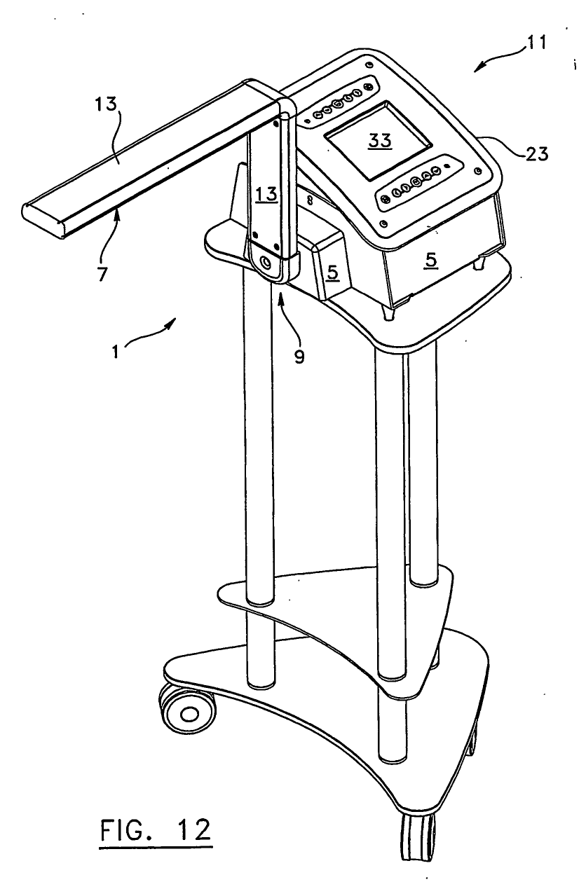

According to another aspect of the present invention, there is also provided an assembly for treating the skin of a patient 3, the assembly comprising a first surface, such as a platform, a bed and the like, for example, for receiving the patient 3, and a second surface, such as a stand for example, adjacent to the first surface, for receiving an apparatus 1 such as the one described herein, for treating the skin of the patient 3 of the first surface with the apparatus, as can be easily understood when referring to FIGS. 1 and 12.

According to yet another aspect of the present invention, there is also provided a method of treating the skin of a patient 3, the method comprising the steps of a) providing an apparatus 1 such as the one described herein; and b) operating the control means 11 for controlling the light emitting sources so that each source emits a given ray of light at a given wavelength, and for controlling the moving means 9 so that the light emitting surface 7 is moved with respect to the skin of the patient so as to treat said skin with the rays of light emitted by the light emitting sources.

Indeed, the present invention relates to a light therapy treatment with an apparatus 1 using a plurality of different light sources emitting multiple wavelengths, preferably seven, combined with a microprocessor-controlled robotic arm 13, for treating the skin of an individual. Indeed, this combination allows the use of multiple wavelengths to be applied in a robotically-controlled manner to the skin. Furthermore, the light intensity and wavelength of the light emitting sources is preferably controlled by the microprocessors of the control means 11 to achieve maximum efficiency in the treatment of various skin conditions.

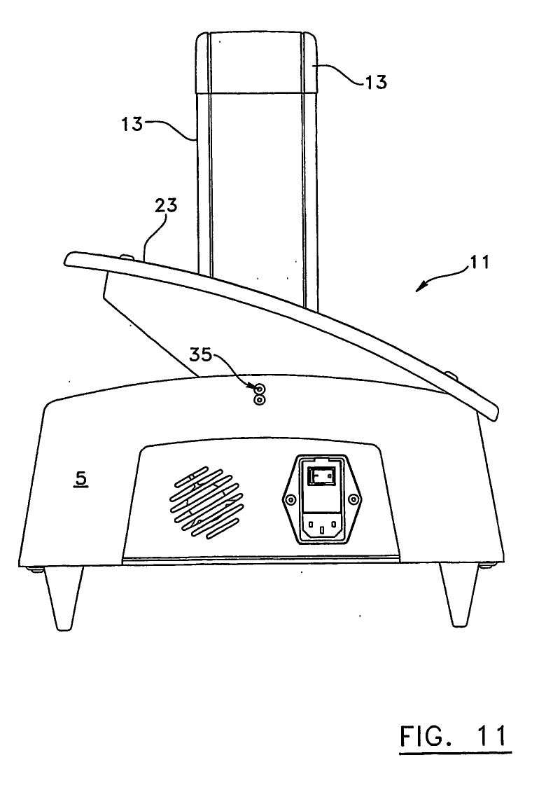

The preferred embodiment of the present invention uses LEDs 21 as light emitting sources. The apparatus 1 preferably uses a plurality of these LEDs 21, preferably one-hundred-eighty, to generate the desired intensity. In the preferred embodiment of the device, the LEDs are positioned in a grid of five rows wide by thirty-six columns long, as can be easily understood from FIG. 11. The LEDs are preferably positioned in a manner that each type of LED is present in only one row of the grid. Therefore, in certain rows, there will be more than one type of LED present. In such case, the LEDs of different types are preferably alternated to ensure a uniform distribution over the length of the light emitting surface 7, which preferably comprises a Printed Circuit Board (PCB). This grid is preferably divided into three sections lengthwise. These sections are preferably controlled independently to vary the light output of each section. Other embodiments of the apparatus according to the present invention could use one or a plurality of such independent sections.

The apparatus 1 preferably uses a motorized arm 13 to displace the light beam on the patient's skin. In the preferred embodiment, the displacement of the arm 13 is radial with a rotational axis 25 aligned with the patient's spine, as better shown in FIG. 2. As previously mentioned, the rotation of the arm 13 is preferably accomplished using a servomotor 15 mated to a planetary gearbox 17. The gearbox 17 is used to reduce the rotational speed of the arm 13 while increasing the torque. The use of the gearbox 17 is preferable but not obligatory. The servomotor 15 is preferably controlled using a microcontroller to ensure precise displacement. Using this combination of servomotor 15 and microprocessor, one can vary the radial speed of the arm 13. With this variation, one can change the time of exposure of certain areas of the skin. This allows the technician to increase the intensity of the treatment in specific areas. Other embodiments for the present invention could use a linear displacement to allow the apparatus 1 (or arm 13) to move the light beam across the patient's skin. This could be done for the head and shoulders or for the whole body for example. Furthermore, the light emitting surface 7 could be shaped in a way that the beam of light will cover part of the body (front or back) or cover the whole body (front and back). The apparatus 1 could be designed so that the patient 3 is positioned horizontally or it could be designed so that the patient is positioned vertically. Furthermore, as previously mentioned, the displacement of the beam of light, or light emitting surface 7, could be simulated by a modulation of the LEDs in a fixed panel. In this type of configuration, the LEDs would be lit row by row in a sequential manner, for example. This modulation could be seen as a wave moving across the fixed panel.

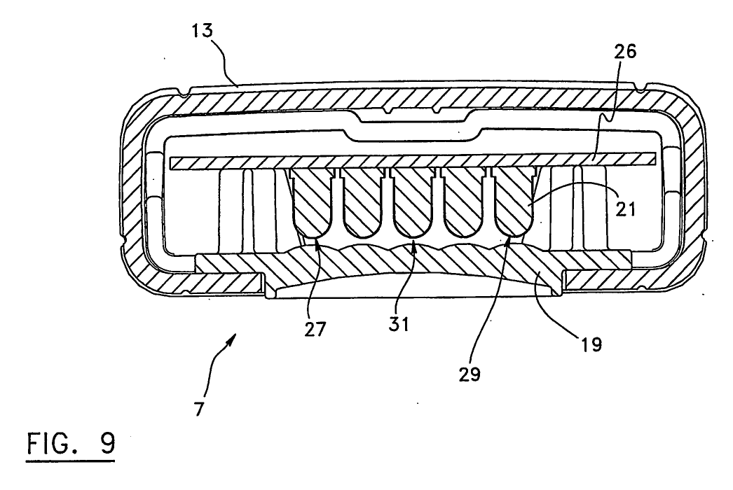

As also previously mentioned, to increase the intensity of the light being emitted, the preferred embodiment of the apparatus 1 employs a specific optical lens 19 shaped and sized to redirect the light emitted from the LEDs. In fact, the top surface of the optical lens 19 is preferably used to redirect the rays of light emitted so that they are made perpendicular to the printed circuit board on which the LEDs are mounted. As can be easily understood from FIG. 9, the bottom surface of the optical lens 19 redirect the rays of light emitted by the LEDs on either side 27, 29 of the PCB so that they will be inline with the center 31 of the PCB. Therefore, the light being emitted by each type of LEDs is concentrated and redirected to achieve maximum treatment intensity. Other embodiments of the invention could use a different optical lens design or no lens 19 at all, for that matter, as apparent to a person skilled in the art.

In the preferred embodiment of the present invention, the control panel 23 and its corresponding LCD display 33 are mounted on a pivot 35. This configuration allows the user to choose the side on which the control panel 23 will be located. The present invention is preferably devised so that by moving the top control panel 23 to either side, the apparatus 1 registers the position of the control panel 23 and will orient the LCD display 33 accordingly. Therefore, if the control panel 23 is tilted to the left, the display 33 will be orientated so that the left side of the display 33 will be the bottom of the screen. Furthermore, if the control panel 23 is tilted to the right, the display 33 will be orientated so that the right side of the display 33 will be the bottom of the screen. Other embodiments of the present invention could use a fixed control panel 23 that would be orientated in a set direction or a mobile control panel 23 that could be moved manually to the desired position using a system consisting of levers and pivots.

As may now be appreciated, the present invention is a substantial improvement over the prior art in that, by virtue of its design and components, the light therapy apparatus 1 is very simple and easy to use, as well as is very simple and easy to manufacture and/or assemble, without compromising the reliability of its functions. Furthermore, by virtue of its design and components, as explained herein, the light therapy apparatus 1 does not require the use of an operator for displacing the light emitting surface 7 about a patient 3. Indeed, the light emitting surface 7 is displaced about the skin to be treated by means of the moving means 9, which preferably consists of a movable arm 13, but which may take on various other embodiments, as briefly explained herein, and as apparent to a person skilled in the art. Furthermore, because moving means 9 are used to displace the light emitting surface 7, different portions of the skin of the patient may be treated alternatively as a result of the light emitting surface 7 sweeping above said portions along a predetermined path and at a predetermined rate. Moreover, because of its moving means 9, the light therapy apparatus according to the present invention does not require the use of pulsating light emitting sources, in that, because the arm 13 is swept across a surface to be treated, the light emitting sources may be kept constantly, thereby prolonging their lifespan. The present invention is also advantageous in that because of its tiltable control panel 23, an operator may easily activate a given program either from the left side or the right side of the base 5. The present invention is also advantageous in that, instead of a fixed light emitting surface placed before the patient to be treated, the light emitting surface 7 according to the present invention is moved about the skin to be treated, thereby enabling a more effective and more enjoyable treatment for the patient 3. Hence, it may now be appreciated that the present invention represents important advantages over other light therapy apparatuses known in the prior art, in terms of performance, versatility and costs.

Of course, numerous modifications could be made to the above-described embodiments without departing from the scope of the invention, as defined in the appended claims.

1. An apparatus for treating the skin of a patient, the apparatus comprising:
a base;
a light emitting surface including an array of light emitting sources, each source being configured for emitting different rays of light at different wavelengths;
moving means for controllably and remotely moving the light emitting surface with respect to the base along a predetermined path and at a predetermined rate; and
control means for controlling the light emitting sources so that each source emits a given ray of light at a given wavelength, and for controlling the moving means so that the light emitting surface is moved to treat the skin of the patient.

2. An apparatus according to claim 1, wherein the moving means comprise a movable arm having a first portion along which extends the light emitting surface, and a second portion having an end pivotally connected to the base.

3. An apparatus according to claim 2, wherein the moving means comprise a servomotor and a gearbox, the gearbox being operatively connected to the end of the arm pivotally connected to the base, and the servomotor being operatively connected to the gearbox for driving the same and thereby moving the arm.

4. An apparatus according to claim 3, where the gearbox is a planetary gearbox.

5. An apparatus according to claim 3, wherein the servomotor is connected to the control means for receiving control signals from the same.

6. An apparatus according to any one of claim 2, wherein the arm is substantially L-shaped.

7. An apparatus according to claim 1, wherein the control means comprises at least one microprocessor.

8. An apparatus according to claim 1, wherein the apparatus comprises an optical lens placed over the light emitting surface, the optical lens being shaped and sized for redirecting the rays of light emitted by the light emitting sources.

9. An apparatus according to claim 8, wherein the optical lens is shaped and sized for redirecting the rays of light emitted by the light emitting sources in a direction substantially perpendicular with respect to the light emitting surface.

10. An apparatus according to claim 1, wherein each light emitting source is configured for emitting rays of light at seven different wavelengths.

11. An apparatus according to claim 1, wherein the array of light emitting sources comprises a least one row and a plurality of transversal columns.

12. An apparatus according to claim 1, wherein the array of light emitting sources comprises five rows and thirty-six columns.

13. An apparatus according to claim 1, wherein the array of light emitting sources comprises a plurality of sections, each section being controllable independently by the control means in order to vary a light output from each section.

14. An apparatus according to claim 13, wherein said plurality of sections includes three sections.

15. An apparatus according to claim 1, wherein the moving means comprise a fixed panel where lies the array of light emitting sources, and wherein the moving means consist of a sequential modulation from the control means of the light emitting sources along the panel for moving the light emitting surface with respect to said panel.

16. An apparatus according to claim 1, wherein the light emitting sources are light emitting diodes.

17. An apparatus according to claim 1, wherein the control means comprises a control panel hingedly connected to a top portion of the base, and being tiltable between opposite left and right sides of the base.

18. An apparatus according to claim 1, wherein the apparatus comprises power receiving means for receiving power from a power supply.

19. An assembly for treating the skin of a patient, the assembly comprising:
a first surface for receiving a patient; and
a second surface, adjacent to the first surface, for receiving an apparatus according to claim 1, for treating the skin of the patient of the first surface with said apparatus.

20. A method of treating the skin of a patient, the method comprising the steps of:
a) providing an apparatus according to claim 1;
b) operating the control means for controlling the light emitting sources so that each source emits a given ray of light at a given wavelength, and for controlling the moving means so that the light emitting surface is moved with respect to the skin of the patient so as to treat said skin with the rays of light emitted by the light emitting sources.
